(19) United States
(12) Patent Application Publication
(10) Pub. No.: US 2011/0306863 A1
(43) Pub. Date: Dec. 15, 2011

(54) PATIENT ALIGNMENT SYSTEM FOR DIAGNOSTIC AND THERAPEUTIC PROCEDURES

(76) Inventors: Jason Koshnitsky, Framingham, MA (US); Alan Sliski, Lincoln, MA (US)

(21) Appl. No.: 12/815,812

(22) Filed: Jun. 15, 2010

(51) Int. Cl. A61B 5/05

(52) U.S. Cl. 600/407

(57) ABSTRACT

A patient alignment system for diagnostic and therapeutic procedures where the embodiment is mounted or referenced to the patient positioning interface such as add-on positioning devices, or directly with the diagnostic and/or therapeutic treatment table or couch. Some patient and equipment positions can obstruct fixed wall or ceiling mounted lasers or an optical view of the anatomy being imaged or treated and patient set-up and alignment may become less accurate or not possible. The patient alignment system may use lasers, cameras or other optical means, ultrasound or RF transceiver technologies, or a combination of multiple technologies, and be mounted in positions, such as below the treatment table or couch and offer a solution to patient alignment for such circumstances. Prone breast imaging and treatment is one example where this system may be used to advantage.

### RELATED APPLICATIONS

U.S. Application No. 20090064413 filed on Sep. 5, 2008 is incorporated reference herein in its entirety.

### BACKGROUND

Accurate patient alignment can be important when imaging and treating patients. In one implementation, such as cancer radiation therapy, reproducibility of the anatomical position determined by diagnostic imaging modalities such as CT, MR and ultrasound can be crucial in accurately delivering the therapeutic dose using external beam radiation.

Optical alignment systems, such as lasers and cameras, can be used for patient set-up. In this example, patient set-up generally involves having at least 3 points of reference, on or in the patient. One technique is to apply tattoos to the patient's skin during the imaging procedure in chosen locations. During subsequent treatment procedures, the tattoos are used in conjunction with wall and/or ceiling mounted lasers to re-align the patient to the same relative position and alignment as during the imaging procedure. The wall and ceiling mounted lasers may be aligned to one virtual point (isocenter) and can guide medical personnel in properly setting up the patient by projecting beams onto the marks previously applied to the patient. Wall and ceiling mounted lasers do not address the needs of some emerging therapies where in one instance the patient or parts of the body are not in the field of view of the alignment system. Prone breast imaging and radiotherapy is one example where wall and ceiling mount lasers and/or optics are not visible on some parts of the anatomy. With the patient in the prone position, it can become uncertain that the breast is in the same position from day to day. For example, the imaging process used in radiotherapy can start with a CT simulation, where the patient is marked. Lasers and/or cameras are used to determine reference points on the skin of the patient. These points can be marked with a permanent-type marker or even tattooed. During the treatment planning process, the relative position of the patient markings to the target volume to be treated is measured. Using these marks at a later time, the imaged/simulated patient set-up can be reproduced for the delivery of therapeutic radiation. This process assumes that the target volume within the patient to be treated does not move relative to the marks on the surface of the patient. It is therefore useful to have the marks on the patient in close proximity to the target volume to minimize the alignment error. Using the prone position breast treatment as an example, the back of the patient may be marked for alignment as it is the only surface of the patient accessible from the ceiling mounted laser. As the mark on the back of the patient is a significant distance from the breast and the anatomy is not rigid, set-up accuracy is not maximized with this approach. Reproducibility can also play an important role in multi-modality imaging where data sets from one modality such as CT are merged with another such as MR, PET, or ultrasound.

### BRIEF DESCRIPTION OF THE DRAWINGS

FIG. 1 is an illustration of one set-up of the alignment system using a single alignment system without being attached to any surfaces.

FIG. 2 is an illustration of another set-up where more than one alignment system can be used.

FIG. 3 is an illustration of an alignment system that can be used in conjunction with an add-on patient positioning system, such as ClearVue™ prone breast table.

FIG. 4 is an illustration of an alignment system that can be used directly with an imaging or treatment couch/table, again using breast an example.

### DETAILED DESCRIPTION

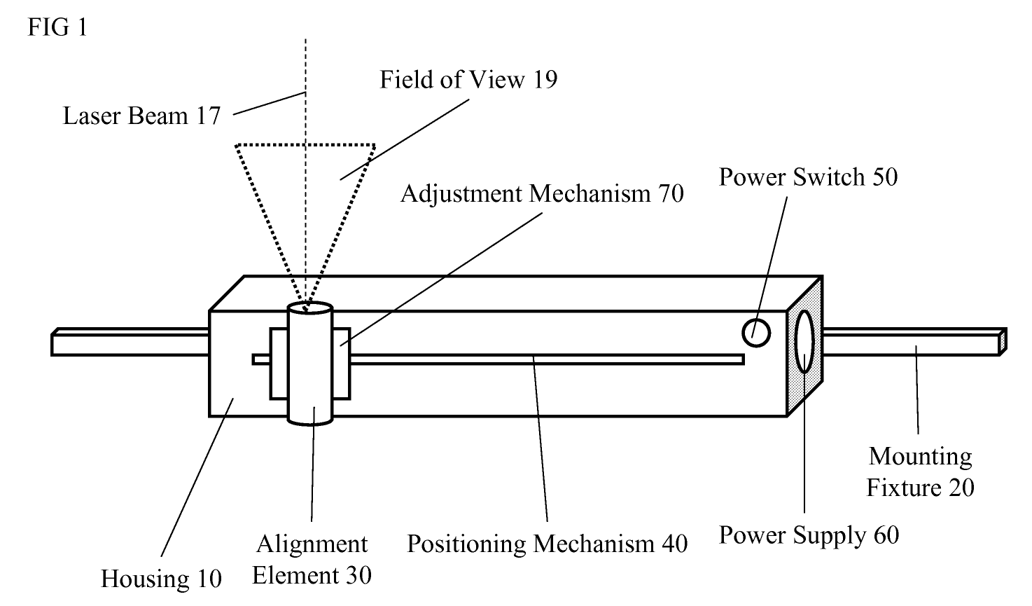

FIG. 1 shows one implementation of an alignment system comprising a housing 10, a mounting fixture 20, an alignment element 30, a positioning mechanism 40, a power switch 50, a power supply 60, and an adjustment mechanism 70. The housing 10 can be made from any suitable material such as metal, plastic wood, composite or the like, and can be a container-like structure of any shape or size. The mounting fixture 20 can be of various shape and size and may have a combination of materials and sub-components such as bolts, screws and the like. The alignment element 30 can be a laser, still camera, video camera, projector, ultrasound device, RF transceiver or the like.

The positioning mechanism 40 can be a rail or any other mechanism allowing motion along one, or more axes and it can be manually or automatically driven and may contain motors. Power switch 50 can be manual, automatic, on/off, timer controlled, or remote controlled. The power supply 60 can be battery or mains powered, and it can be located on the device or separately. The adjustment mechanism 70 can be made from any stable material such as metal, plastic or the like and can include pivots, screws, ball joints or the like. The alignment system may employ multiple fixed alignment elements instead of one moveable unit.

The housing 10 may function as the container for the alignment element 30 and can hold or contain any or all of the described elements. The mounting fixture 20 may function as the mounting piece to attach the alignment system to add-on patient positioning devices, patient exam tables, treatment couches, diagnostic couches, or any other stable material. The alignment element 30 can act as an emitter, such as a laser or projector, to emit for an example a laser beam 17 or an alignment pattern within field of view 19. The alignment element 30 can also act as a receiver, such as a camera, or be a combination of both the transmitter and a receiver.

The positioning mechanism 40 may function to allow the movement of the alignment element 30 in any direction along all 3 axes. The power switch 50 may function to turn the system power on an off. It can be a toggle, push button or the like, and can be activated at the device or remotely. It can be timer controlled to remain on for a certain period of time. The power supply 60 can power the system and include a timer to turn the alignment element 30 on and off. The adjustment mechanism 70 may rotate along all axes to move or rotate the alignment element 30. The housing 10 may act as a container to contain or hold together the alignment element 30, positioning mechanism 40, power switch 50, power supply 60, mounting fixture 20, adjustment mechanism 70 and the like. The mounting fixture 20 can be permanently or temporarily connected to housing 10, and can vary for different mounting options depending on placement of the alignment system. The alignment element 30 can be mounted onto the adjustment mechanism 70 or it can be mounted on the housing 10 or it can be mounted on the positioning mechanism 40. The adjustment mechanism 70 can be mounted to the alignment element 30 and can move the alignment element 30 along all axes and directions to point the alignment element 30 in the desired direction. The positioning mechanism 40 may have mountings for the adjustment mechanism 70 or alignment element 30. The positioning mechanism 40 may allow the alignment element 30 and/or adjustment mechanism 70 to move along one or more axes. The power switch 50 can be connected to the power supply 60 and function to turn the system on and off. The power supply 60 can be connected to provide power to the alignment element 30, adjustment mechanism 70, positioning mechanism 40 and other elements requiring power. One activated by the user, the power supply 60 would supply power to the active alignment elements 30 enabling the user to mark or record the patient position information during the imaging procedure, and later, during the patient set-up for the therapy procedure, allow the alignment elements 30 to illuminate, display, or measure the marked or recorded patient position information to facilitate correct patient anatomy positioning.

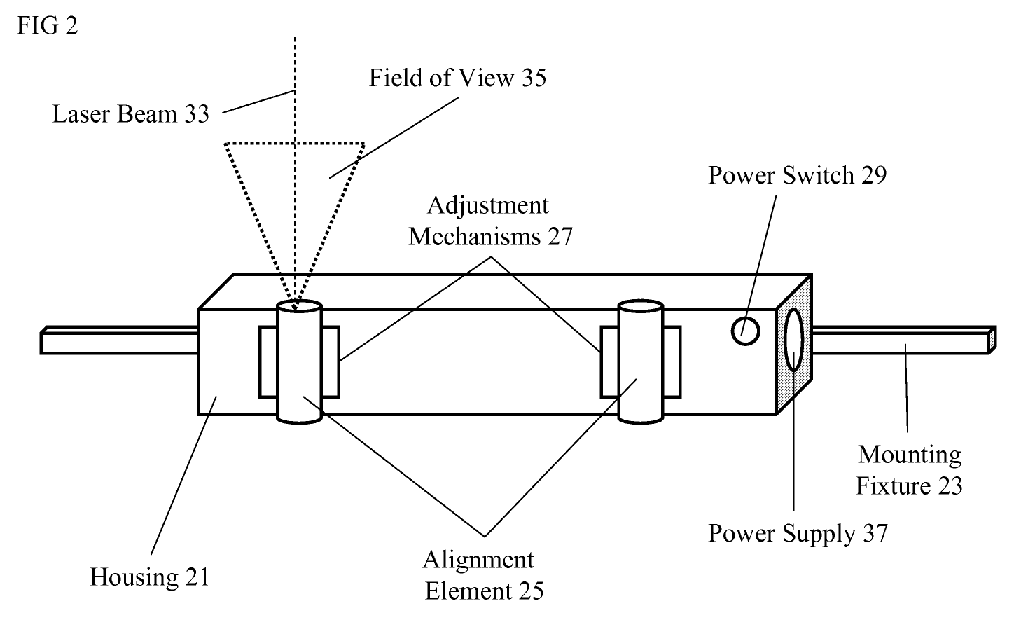

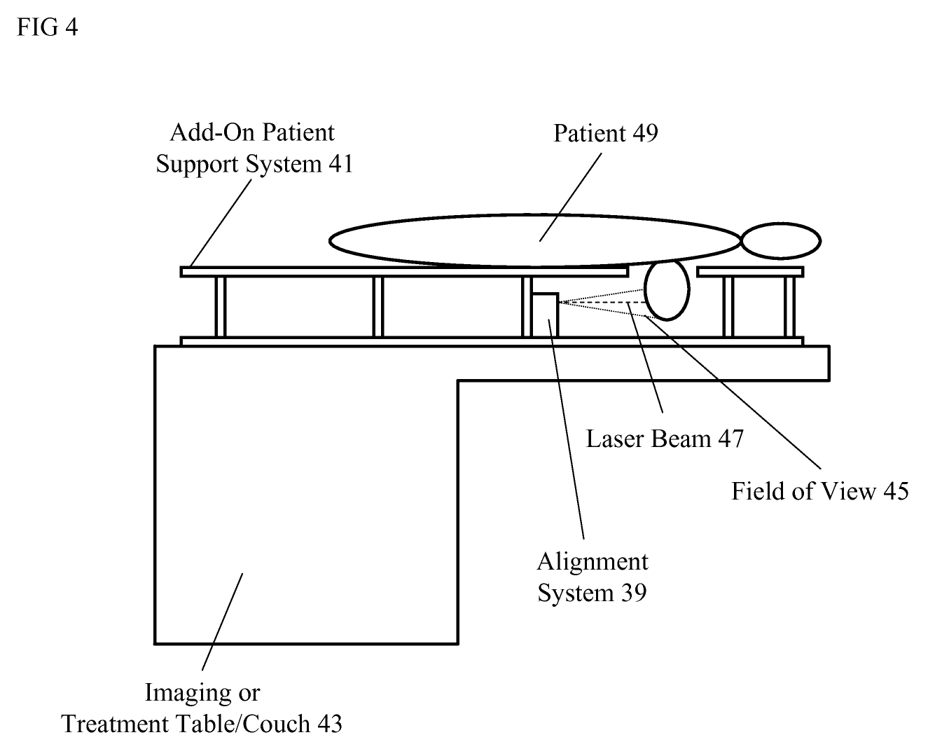

FIG. 2 describes one implementation of an alignment system comprising a housing 21, a mounting fixture 23, more than one alignment element 25, a power control 29, a power supply 37, and adjustment mechanism 27. More than one alignment element can be employed for the projection of multiple crossing laser lines, for example, or a plurality of transceiver elements for use with radio frequency surface markers or implants. The housing 21 can be made from any stable material such as metal, plastic, wood, composite or the like, and can be a container-like structure of any shape or size. The mounting fixture 23 can be of various shape and size and may have a combination of materials and sub-components such as bolts, screws and the like. The alignment elements 25 can be laser, still camera, video camera, projector, ultrasound device, RF transceiver or the like. Power switch 29 can be manual, automatic, on/off, timer controlled, or remote controlled. The power supply 37 can be battery or mains powered, and it can be located on the device or remotely. The adjustment mechanism 27 can be made from any stable material and can include pivots, screws, ball joints or the like. The housing 21 can function as the container for the alignment system and can hold or contain any or all of the described elements. The mounting fixture 23 can function as the mounting piece to attach the alignment system to add-on patient positioning devices, patient exam tables, treatment couches, diagnostic couches, or any other stable material. The alignment elements 25 can act as emitters, such as a laser or projector, to emit for an example a laser beam 33 or a projected image within a field of view 35. The alignment elements 25 can also act as receivers, such as a camera, or be a combination of both the transmitter and a receiver. The alignment system can be configured to record images of the anatomy at one time, then project the same or other image at another time to facilitate alignment of the anatomy from day to day. The power switch 29 may function to turn the system power on an off. It can be a toggle, push button or the like, and can be activated at the device or remotely. It can be timer controlled to remain on for a certain period of time. The power supply 37 can power the system on and off. The adjustment mechanisms 27 may rotate along all axes to move or rotate the alignment elements 25. The housing 21 may act as a container to contain or hold together the alignment elements 25, power switch 29, power supply 37, mounting fixture 23, adjustment mechanisms 27 and the like. The mounting fixture 23 can be permanently or temporarily connected to housing 21, and can vary for different mounting options depending on placement of the alignment system. The alignment elements 25 can be mounted onto the adjustment mechanisms 27 or it can be mounted on the housing 21. The adjustment mechanisms 27 can be mounted to the alignment elements 25 and can move the alignment element along all axes and directions to point the alignment element in the desired direction. The adjustment mechanisms 27 can be manual, motorized, or automated and programmable locally or remotely. The power switch 29 can be connected to the power supply 37 and function to turn the system on and off. The power supply 37 can be connected to provide power to the alignment elements 25, adjustment mechanisms 27, and other elements requiring power. In one implementation of FIG. 2, where the alignment elements are line-projecting lasers, one horizontal line can be projected which is wide enough to illuminate the left or right breast, or both. Two vertical line projecting lasers can be used, one that aligns with the left breast, and one for the right breast. In use, during the imaging procedure, the breast is marked at the intersection of the two crossing laser lines. Later, during the therapy procedure, the patient position is adjusted so that the mark is again aligned to the crossed laser lines. In one implantation of FIG. 2, multiple cameras are mounted to provide imaging capability in one or more directions for each breast. The outline of the patient anatomy and any surface features or marks can be recorded at the time of the imaging procedure. During patient set-up for the therapy procedure, the previously acquired images can be displayed along with the real-time images in order to show the magnitude and direction of the motion required to move the patient into alignment with the earlier reference images. The required motion can be computed using algorithms commonly used in machine vision for inspecting and positioning parts. In one implementation of FIG. 2, the alignment elements consist of co-axial image recording and projection devices. During the imaging procedure, an image of the patient anatomy and any surface features or marks can be recorded with the image recording device. During the later therapy or imaging procedure, the projector can project the same image recorded earlier or any other image from the same point of view to assist in the repositioning of the patient for improved alignment of the target volume to the treatment modality. In one implementation of FIG. 2, the alignment elements consist of transceivers that can interface with radio frequency transponders on the patient's skin or implanted transponders within the patient's anatomy. These transponders can also be visible in the anatomical views generated during the imaging procedure.

FIG. 3 shows one implementation of the patient alignment system 39, where it can be used with add-on patient support system 41 that can be used in conjunction with imaging or treatment table/couch 43. The patient alignment system 39 can be comprised of the system described in FIG. 1 and FIG. 2. The imaging or treatment table/couch 43 can be an examination table, CT table, ultrasound table, MRI table or couch, particle beam therapy table or couch, or the like. The add-on support system can be a patient positioning device, such as the ClearVue™ prone breast table, or the like. The imaging or treatment table/couch 43 can be used to position and examine the patient, obtain diagnostic images, perform biopsies and/or surgical procedures, deliver radiotherapy treatment, deliver hyperthermia treatment, or the like. The add-on patient support system 41 can be used to position the patient 49 in a unique way, for example placing a breast patient in the prone position. The alignment system 39 can be used to align the patient 49 using a projector or emitter such as the laser beam 47, or receiver, such as the optical camera 45. The add-on patient support system 41 may be placed on top of the imaging or treatment table/couch 43. The patient 49 can be positioned on top of the add-on support system 41, and the alignment system 39 can be used to align the patient 49 to the desired position. After the patient is positioned on the support system, the imaging study can be conducted. In conjunction with the imaging study, the alignment system 39 can be activated and the patient marked on the surface or profile and surface markings acquired for contemporaneous or later use. The patient alignment system may also determine and record the position of surface or implanted RF tags at any time during the imaging session. The position information is recorded on the patient through marking, electronically through image or marker position acquisition and stored

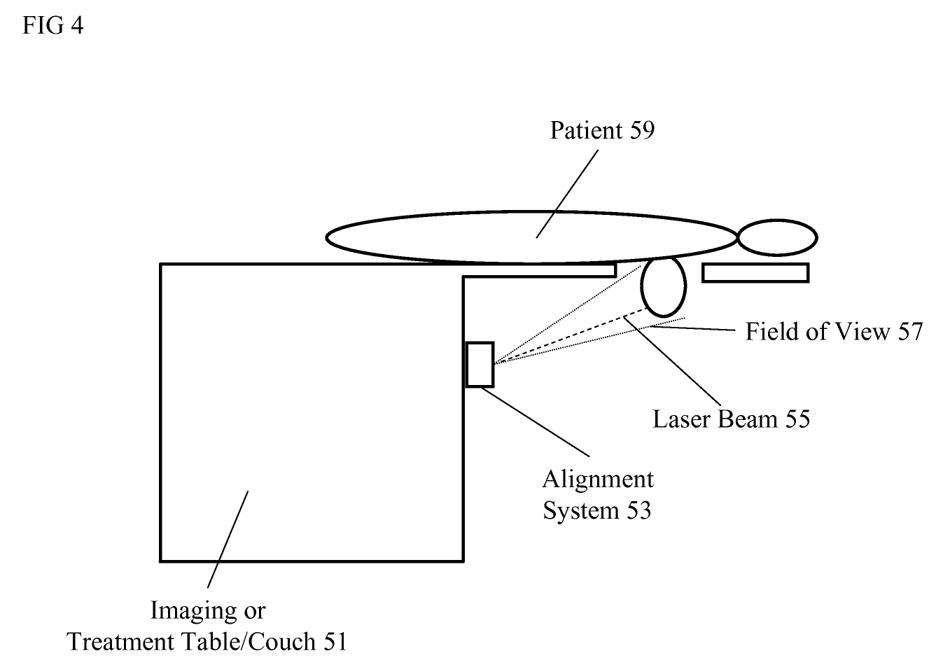

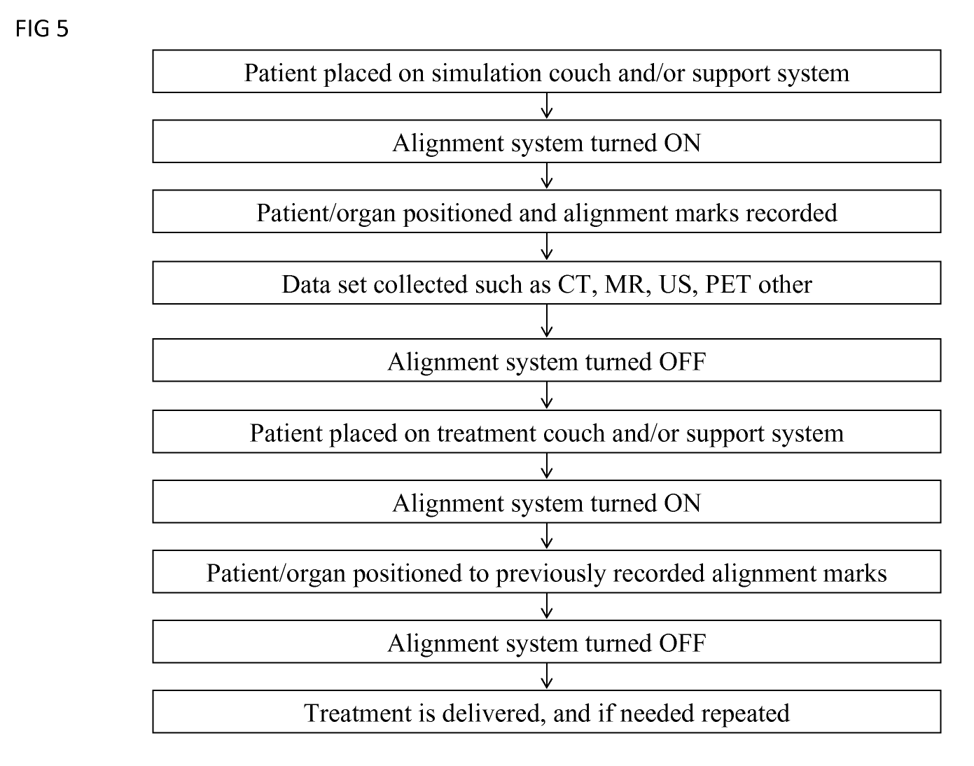

FIG. 4 shows one implementation of the patient alignment system 53, where it can be used as an integrated part of the imaging or treatment table/couch 51. The patient alignment system 53 can be comprised of the system described in FIG. 1 and FIG. 2. The imaging or treatment table/couch 51 can be an examination table, CT table, ultrasound table, or MRI table or couch, particle beam therapy table or couch, or the like. The imaging or treatment table/couch 51 can be used to examine the patient, obtain diagnostic images, perform biopsies and/or surgical procedures, deliver radiotherapy treatment, deliver hyperthermia treatment, or the like. The alignment system 53 can be used to align the patient 59 using a projector or emitter such as the laser beam 55, or receiver, such as the optical camera 57. The patient 59 can be positioned on top of the imaging or treatment couch/table 51, and the alignment system 53 can be used to align the patient 59 to the desired position.

1. A patient alignment system comprising:
a. One or more active alignment elements attached or registered to a patient interface surface;
b. A housing and mounting system for the alignment elements;
c. Power control for the active alignment elements.

2. A patient alignment system can be mounted or referenced to an add-on positioning system or existing diagnostic or treatment patient support

3. A patient alignment system can project an optical alignment pattern.

4. A patient alignment system can acquire, store, retrieve, and display an optical image.

5. A patient alignment system can transmit or receive ultrasonic acoustic information.

6. A patient alignment system can use a radio frequency transceiver.

7. A patient alignment system where the alignment elements can be a combination of more than one technology, such as optical and ultrasonic combined.

8. The alignment system in where the alignment element can be an optical camera and/or projector.

9. A patient alignment system where the controls can be mounted directly on or remotely from the alignment system.
